(19) United States
(12) Patent Application Publication
(10) Pub. No.: US 2015/0346185 A1
(43) Pub. Date: Dec. 3, 2015

(54) DIAGNOSIS OF CYSTIC FIBROSIS

(71) Applicants: INSTITUT NATIONAL DE LA SANTE ET DE LA RECHERCHE MEDICALE (INSERM), Paris (FR); UNIVERSITE PARIS-EST CRETEIL VAL DE MARNE, Créteil Cedex (FR); CENTRE HOSPITALIER INTERCOMMUNAL DE CRETEIL, Creteil (FR)

(72) Inventor: Virginie PRULIERE-ESCABASSE, Creteil (FR)

(21) Appl. No.: 14/648,785

(22) PCT Filed: Dec. 5, 2013
(86) PCT No.: PCT/EP2013/075694

§ 371 Date: Jun. 1, 2015

(30) Foreign Application Priority Data
Dec. 5, 2012 (EP) 12306523.7

(51) Int. Cl. G01N 33/483; G01N 27/04

(52) U.S. Cl. CPC G01N 33/4833; G01N 27/04

(57) ABSTRACT

The present invention provides materials and methods for diagnosis of cystic fibrosis, including atypical cystic fibrosis.

### FIELD OF THE INVENTION

The present invention provides materials and methods for diagnosis of cystic fibrosis, including atypical cystic fibrosis.

### BACKGROUND TO THE INVENTION

Cystic Fibrosis (CF) is the most common genetic disease in Caucasian countries, with an incidence of 1 birth in 3000. It is an autosomal recessive disease linked to mutations in the CFTR gene whose nature determines the clinical expression and severity of the disease, affecting mainly the respiratory, digestive and genital systems.

The respiratory disease is mainly responsible for the morbidity and mortality in patients with cystic fibrosis. CFTR, a chloride-ion channel, plays a critical role in lung disease through its involvement in the changes of surface liquid covering airway epithelial cells. Dehydration of the surface liquid leads to altered mucociliary clearance and inflammation and infections at the mucosal epithelia.

Since the cloning of the CFTR gene in 1989, over 2000 mutations of the gene have been described. The F508 mutation (deletion of phenylalanine at position 508 of the protein) is the most frequent (70% of mutated alleles in patients with CF). The different CFTR mutations may result in lack of expression of the gene, defects in maturation of the protein, lack of incorporation of the protein at the apical membrane of epithelial cells or functional abnormalities of the chloride channel. The F508 mutation is responsible for a failure of maturation of the CFTR protein, leading to its partial destruction in the cell. Functionally, epithelial transport of chlorine and sodium are disrupted, with sodium absorption by airway epithelial cells three times that of a normal epithelium. In normal epithelium, the transepithelial nasal potential difference is approximately −30 mV, mainly due to the active absorption of sodium (more than 50% of the Na⁺ conductance).

The major Na⁺ channel in the upper airway is the amiloride-sensitive channel ENaC. In cystic fibrosis, nasal transepithelial potential difference measured is increased in patients (≧40 mV), chloride secretion in the apical membrane is reduced and weakly activated by cyclic AMP (as opposed to healthy subjects).

The pathophysiology of respiratory disease in CF patients is related to the consequences of the absence or dysfunction of CFTR due to the reduction in membrane permeability of epithelial cells to Cl⁻. Moreover, the removal of the inhibition normally exerted by CFTR on ENaC induced hyperabsorption of Na⁺. The hyperabsorption of Na⁺ and water associated with defective secretion of Cl⁻ ions then reduces the height of the liquid covering the hair cells of the respiratory system (5). The hyperabsorption of the Na⁺ channel ENaC has been demonstrated in patients with cystic fibrosis by infusion of its specific inhibitor amiloride, which reduced their nasal transepithelial potential difference of 75 to 90% against only 55% in patients healthy.

Diagnosis of cystic fibrosis (CF) is usually made by the presence of sinopulmonary disease, which may be associated with pancreatic deficiency, with abnormal sweat chloride values (sweat chloride secretion>60 mmol/l) and/or the finding of two cystic fibrosis transmembrane conductance regulator (CFTR) mutations. However, an emerging number of patients present with an atypical phenotype of the disease may have normal or intermediate range sweat chloride level, between 30 and 59 mmol/l, and only one or no identified CF-causing mutations. Despite the cloning of the CFTR gene and the identification of more than 700 CFTR gene mutations, routine genetic analysis can not confirm the diagnosis of CF caused by rare or unidentified CFTR gene mutations. Thus, the diagnosis of cystic fibrosis (CF) is not always certain, despite extensive clinical evaluation, multiple sweat chloride tests and genotype analysis.

Currently, investigators use nasal potential difference (DPN) measurements to demonstrate abnormal function of the cystic fibrosis transmembrane conductance regulator and establish a diagnosis of CF in patients with atypical presentations. However, DPN is an invasive procedure requiring the insertion of an electrode inserted into the nasal mucosa and a second electrode inserted into the arm. This is particularly problematic as the existence of rhino-sinusal infections in many CF patients can render such a test impossible. In addition, there is significant variation of DPN measurements between centers with respect to many aspects of the technique (in particular, the type of electrodes used, the temperature of perfusate, the dose of amiloride and the area of the nasal cavity). DPN can also be affected by a number of parameters, including exercise and drugs, though principally by chronic inflammation in CF upper airways.

Early diagnosis of CF is important as it may permit treatment to be commenced before the apparition of irreversible lesions. There is thus a need for alternative methods of diagnosis of CF, in particular in cases where no CFTR mutations are present, such as CF associated with an atypical phenotype.

### DESCRIPTION OF THE INVENTION

The inventors have developed a new ex vivo test for the diagnosis of CF patients. After local anesthesia, they collected human nasal epithelial cells (HNEC) by nasal brushing and cultured them at air-liquid interface. Chloride and sodium transport in the cultured HNEC were evaluated in Ussing chambers by short-circuit current measurements. The results demonstrated that this method could be used to effectively demonstrate differences between short-circuit current measurements in HNEC cells from cystic fibrosis patients compared to control subjects. The method further allows distinguishing cystic fibrosis patients having a classic phenotype from those having an atypical phenotype.

The method according to the invention has the advantages of being non invasive, since the test sample may originate from nasal epithelial cells obtained by nasal brushing. Moreover, contrary to the DPN measurements, the diagnostic method according to the invention may be used for any patients, whatever their physical condition is.

Cystic fibrosis is traditionally diagnosed by the presence of at least one major clinical feature (typical pulmonary or gastrointestinal manifestations) or a family history of CF, accompanied by either two or more sweat chloride measurements greater than 60 mmol/l or by CF-causing gene mutations on both chromosomes.

By “a cystic fibrosis patient having a classic phenotype”, it is meant herein a patient having a sweat chloride secretion higher than 60 mmol/l and/or at least two cystic fibrosis transmembrane conductance regulator (CFTR) CF-causing mutations.

The diagnostic method according to the invention is very useful for diagnosing patients suffering from cystic fibrosis with an atypical phenotype.

By “a cystic fibrosis patient having an atypical phenotype”, it is meant herein a patient having a sweat chloride secretion equal or lower than 59 mmol/l, for example comprised between 30 mmol/l and 59 mmol/l and/or having only one or no identified cystic fibrosis transmembrane conductance regulator (CFTR) CF-causing mutation.

The inventors have shown that cystic fibrosis patients with an atypical phenotype have abnormal Isc and/or ΔVte values. The diagnosis method according to the invention allows distinguishing an atypical phenotype associated with an altered chloride transport, as well as an atypical phenotype associated with an altered sodium transport.

Non-limiting examples of CF-causing mutations are ΔF508 and 5-Thymidine allele in intron 8 (IVS8).

ΔF508 (delta-F508, full name CFTRΔF508 or F508del-CFTR; rs113993960) is the most frequent mutation within the gene for CFTR protein. The mutation is a deletion of the three nucleotides that comprise the codon for phenylalanine (F) at position 508. A person with the CFTRΔF508 mutation produces an abnormal CFTR protein that lacks this phenylalanine residue. This protein does not escape the endoplasmic reticulum for further processing, which correspond to a class 2 mutation.

The 5-Thymidine allele in intron 8 (IVS8) of the CFTR gene causes abnormal splicing in the CFTR gene and corresponds to a class 5 mutation. The 5-Thymidine allele in intron 8 (IVS8) is associated with lung disease when it occurs in cis with a missense mutation in the CFTR gene, such as the mutation R117H. However, the 5-Thymidine allele in intron 8 (IVS8) alone may cause a low level of full-length functional CFTR protein and CF-like lung disease.

By “healthy individual”, it is meant herein an individual who does not suffer from cystic fibrosis. More particularly, a healthy individual has a normal sweat chloride secretion and no identified CF-causing mutation. A normal sweat chloride secretion corresponds to a sweat chloride secretion lower than 30 mmol/l.

Sweat chloride secretion may be measured by any method well known in the art.

The expressions “human nasal epithelial cells”, “HNEC” and “HNEC cells” are herein synonymous.

The method of diagnosis according to the invention may be an ex vivo and/or an in vitro method of diagnosis.

The terms “ENAC” and “ENaC” are herein synonymous.

For the first time, the inventors have shown that measuring only the cAMP dependent component of the basal short circuit current (I_{sc cAMP}) in a test sample of human nasal epithelial cells allows detecting almost all cases of cystic fibrosis patients.

Thus, in one aspect, the invention relates to a method of diagnosis of cystic fibrosis, the method comprising measuring in a test sample of human nasal epithelial cells (HNEC), preferably obtained by culturing cells collected by nasal brushing, the cAMP dependent component of the basal short circuit current (I_{sc cAMP}). The I_{sc cAMP} measured value may then be compared to a normal control.

As used herein, a “normal control” may refer to a value measured in a control sample that originates from a healthy individual, said control sample and the test sample being prepared and measured in the same conditions, to a predetermined value or to a predetermined range of values.

When referring to a normal control, the expressions “value”, “normal value” and “normal control value” are synonymous.

When referring to a normal control, the expressions “range of values”, “normal range of values” and “normal control range of values” are synonymous.

In a preferred embodiment, a “normal control” refers to a predetermined value or range of values.

An example of predetermined value for I_{sc cAMP} is 2 μA/cm².

An example of predetermined range of values for I_{sc cAMP} is from 6 to 10 μA/cm².

The present invention particularly relates to a method of diagnosis of cystic fibrosis comprising:
measuring in a test sample of human nasal epithelial cells (HNEC), preferably obtained by culturing cells collected by nasal brushing, the cAMP dependent component of the basal short circuit current (I_{sc cAMP}), and
deducing that said test sample originates from a cystic fibrosis patient when I_{sc cAMP} is below 2 μA/cm².

The present invention also relates to a method of diagnosis of cystic fibrosis comprising:
measuring in a test sample of human nasal epithelial cells (HNEC), preferably obtained by culturing cells collected by nasal brushing, the cAMP dependent component of the basal short circuit current (I_{sc cAMP}),
comparing said I_{sc cAMP} measured value to a normal control, and
making a diagnosis based on said comparison, wherein a diagnosis of cystic fibrosis is made when I_{sc cAMP} is lower in the test sample than the normal control.

Furthermore, the inventors have shown that the measure of the epithelial Na⁺ channel (ENaC) dependent component of the basal short circuit current (I_{sc ENAC}), the measure of the basal short circuit current (I_{sc basal}) and the measure of the transepithelial potential difference (ΔVₜₑ) each allows further deducing if said test sample originates from a cystic fibrosis patient with a classic phenotype or from a cystic fibrosis patient with an atypical phenotype.

The present invention thus also relates to a method of diagnosis as defined above, the method further comprising measuring in a test sample of human nasal epithelial cells (HNEC), preferably obtained by culturing cells collected by nasal brushing:
(a) the epithelial Na⁺ channel (ENaC) dependent component of the basal short circuit current (I_{sc ENAC}),
(c) the basal short circuit current (I_{sc basal}), and/or
(d) the transepithelial potential difference (ΔVₜₑ).

The I_{sc ENAC}, I_{sc basal} and/or ΔVₜₑ measured values may then be compared to a normal control.

An example of predetermined value for I_{sc ENAC} is 20 μA/cm² or 30 μA/cm².

An example of predetermined range of values for I_{sc ENAC} is from 10 μA/cm² to 22 μA/cm².

An example of predetermined value for I_{sc basal} is 30 μA/cm² or 40 μA/cm².

An example of predetermined range of values for I_{sc basal} is from 23 μA/cm² to 37 μA/cm².

An example of predetermined value for ΔVₜₑ is 25 mV or 30 mV.

An example of predetermined range of values for ΔVₜₑ is from 15 mV to 30 mV.

The method may comprise measuring in a test sample of human nasal epithelial cells (HNEC):
(a) the cAMP dependent component of the basal short circuit current (I_{sc cAMP}); and optionally
(b) the epithelial Na⁺ channel (ENaC) dependent component of the basal short circuit current (I_{sc ENAC}); and optionally
(c) the basal short circuit current (I_{sc basal});
and optionally
(d) the transepithelial potential difference (ΔVₜₑ).

The method of diagnosis as defined above may further comprise making a diagnosis based on the measured values,
wherein a diagnosis of cystic fibrosis with a classic phenotype is made when:
   (a) I_{sc cAMP} is below 2 μA/cm², and
   (b) I_{sc ENAC} is above 30 μA/cm², I_{sc basal} is above 40 μA/cm² and/or ΔVₜₑ is above 30 mV, and
wherein a diagnosis of cystic fibrosis with an atypical phenotype is made
   when:
   (a) I_{sc cAMP} is below 2 μA/cm², and
   (b) I_{sc ENAC} is below 20 μA/cm², I_{sc basal} is below 30 μA/cm² and/or ΔVₜₑ is below 25 mV,
   or when:
   (a) I_{sc cAMP} is above 2 μA/cm², and
   (b) I_{sc ENAC} is above 30 μA/cm², I_{sc basal} is above 40 μA/cm² and/or ΔVₜₑ is above 30 mV.

A diagnosis of cystic fibrosis with an atypical phenotype associated with abnormal chloride transport is made when the measured values are:
(a) I_{sc cAMP} below 2 μA/cm², and
(b) I_{sc ENAC} below 20 μA/cm², I_{sc basal} below 30 μA/cm² and/or ΔVₜₑ below 25 mV.

A diagnosis of cystic fibrosis with an atypical phenotype associated with abnormal sodium transport is made when the measured values are:
(a) I_{sc cAMP} above 2 μA/cm², and
(b) I_{sc ENAC} above 30 μA/cm², I_{sc basal} above 40 μA/cm² and/or ΔVₜₑ above 30 mV.

The method may comprise:
(i) measuring in a test sample of human nasal epithelial cells (HNEC):
   (a) the cAMP dependent component of the basal short circuit current (I_{sc cAMP}); and optionally
   (b) the epithelial Na⁺ channel (ENaC) dependent component of the basal short circuit current (I_{sc ENAC}); and optionally
   (c) the basal short circuit current (I_{sc});
   and optionally
   (d) the transepithelial potential difference (ΔVₜₑ);
(ii) making a diagnosis based on said I_{sc} values, and optionally said ΔVₜₑ value; wherein a diagnosis of cystic fibrosis is made when:
   (a) I_{sc cAMP} is below 2 μA/cm²; and/or
   (b) I_{sc ENAC} is above 30 μA/cm²; and/or
   (c) I_{sc basal} is above 40 μA/cm²; and/or
   (d) ΔVₜₑ is above 30 mV.

In another embodiment, the method of diagnosis of cystic fibrosis may comprise:
(i) comparing said I_{sc} measured values, and optionally said ΔVₜₑ measured value, to a normal control, and
(ii) making a diagnosis based on said comparison,
   wherein a diagnosis of cystic fibrosis with a classic phenotype is made when:
      (a) I_{sc cAMP} is lower in the test sample than the normal control, and
      (b) I_{sc ENAC}, I_{sc basal} and/or ΔVₜₑ is higher in the test sample than the normal control,
   and
   wherein a diagnosis of cystic fibrosis with an atypical phenotype is made
      when:
      (a) I_{sc cAMP} is lower in the test sample than the normal control, and
      (b) I_{sc ENAC}, I_{sc basal} and/or ΔVₜₑ is equal or lower in the test sample than the normal control,
      or when:
      (a) I_{sc cAMP} is equal or higher in the test sample than the normal control, and
      (b) I_{sc ENAC}, I_{sc basal} and/or ΔVₜₑ is higher in the test sample than the normal control.

A diagnosis of cystic fibrosis with an atypical phenotype associated with abnormal chloride transport is made when the measured values are:
(a) I_{sc cAMP} lower in the test sample than the normal control, and
(b) I_{sc ENAC}, I_{sc basal} and/or ΔVₜₑ equal or lower in the test sample than the normal control.

A diagnosis of cystic fibrosis with an atypical phenotype associated with abnormal sodium transport is made when the measured values are:
(a) I_{sc cAMP} equal or higher in the test sample than the normal control, and
(b) I_{sc ENAC}, I_{sc basal} and/or ΔVₜₑ higher in the test sample than the normal control.

The method may comprise:

(i) measuring in a test sample of human nasal epithelial cells (HNEC):
(a) the cAMP dependent component of the basal short circuit current (I_{sc cAMP}); and optionally
(b) the epithelial Na⁺ channel (ENaC) dependent component of the basal short circuit current (I_{sc ENAC}); and optionally
(c) the basal short circuit current (I_{sc basal});
and optionally
(d) the transepithelial potential difference (ΔVₜₑ);

(ii) comparing said I_{sc} values, and optionally said ΔVₜₑ value, to a normal control; and

(iii) making a diagnosis based on the comparison,
wherein a diagnosis of cystic fibrosis is made when:
   (a) I_{sc cAMP} is lower in the test sample than the normal control; and/or
   (b) I_{sc ENAC} is higher in the test sample than the normal control; and/or
   (c) I_{sc basal} is higher in the test sample than the normal control; and/or
   (d) ΔVₜₑ is higher in the test sample than the normal control.

Said normal control may be, for example, a predetermined value or range of values, wherein said normal range is optionally:
(a) I_{sc cAMP} of 6-10 μA/cm²;
(b) I_{sc ENAC} of 10-22 μA/cm²; and/or
(c) I_{sc basal} of 23-37 μA/cm².

A preferred range of values for I_{sc cAMP} is from 6 to 10 μA/cm².

A preferred range of values for I_{sc ENAC} is from 10 to 22 μA/cm².

A preferred range of values for I_{sc basal} is from 23 to 37 μA/cm².

A preferred range of values for ΔVₜₑ is from 15 to 30 mV.

For example, the present invention relates to a method as defined above, wherein said normal control is a predetermined value or range of values, wherein said range of values is optionally:
(a) I_{sc cAMP} of 6-10 μA/cm²,
(b) I_{sc ENAC} of 10-22 μA/cm²,
(c) I_{sc basal} of 23-37 μA/cm², and/or
(d) ΔVₜₑ of 15-30 mV.

Measurement of I_{sc} values may be performed by any method known in the art. For example, I_{sc cAMP} may be assayed by stimulation with forskolin and IBMX (I_{sc forsk+IBMX}); and/or I_{sc ENAC} may be assayed by measuring the amiloride sensitive component of the basal short circuit current (I_{sc amil}). In a preferred embodiment, I_{sc cAMP} is assayed, after inhibition of the sodium channels, via a stimulation with forskolin and IBMX (I_{sc forsk+IBMX}); and/or I_{sc ENAC}, where assayed, is assayed by measuring the amiloride sensitive component of the basal short circuit current (I_{sc amil}).

Said test sample may be, for example,
(i) a sample of HNEC cells obtained from an individual suspected of suffering from cystic fibrosis, or
(ii) a sample of HNEC cells obtained by culturing HNEC cells obtained from an individual, preferably by nasal brushing.

In a preferred embodiment, said test sample of human nasal epithelial cells (HNEC) is a sample of human nasal epithelial cells obtained from an individual suspected of suffering from cystic fibrosis.

Said test sample of human nasal epithelial cells (HNEC) is preferably a sample of human nasal epithelial cells obtained by culturing human nasal epithelial cells from an individual suspected of suffering from cystic fibrosis, said cultured cells being preferably obtained by nasal brushing.

The human nasal epithelial cells (HNEC) of said test sample are preferably human nasal epithelial cells obtained by the method for preparing human nasal epithelial cells, as defined below.

In some embodiments, the cells in said test sample have been cultured at an air-liquid interface for at least 14 days prior to the assay, or for at least 5, 6, 7, 8, 9, 10, 11, 12, 13, 14, 15, 16, 17, 18, 19, 20 or 21 days.

Said HNEC cells obtained from an individual may be obtained by brushing of the nasal inferior turbinates, preferably after local anesthesia. Nasal brushing presents the advantages of being safe and painless. Nasal brushing is not a biopsy. This technique allows recovering the three types of nasal epithelial cells (ciliated cells, basal cells and mucous cells). Primary cultures are made from said cells collected by nasal brushing.

The invention also provides a method of monitoring the efficacy of treatment of cystic fibrosis, the method comprising:

(i) measuring in a test sample of human nasal epithelial cells (HNEC) from a cystic fibrosis patient:
(a) the cAMP dependent component of the basal short circuit current (I_{sc cAMP}); and optionally
(b) the epithelial Na⁺ channel (ENAC) dependent component of the basal short circuit current (I_{sc ENAC}); and optionally
(c) the basal short circuit current (I_{sc basal});
and optionally
(d) the transepithelial potential difference (ΔVₜₑ)

wherein said sample is (I) a sample from a patient prior to or during said treatment, and (II) a sample or samples taken from said patient at a later time point during said treatment, or after receiving said treatment; and comparing the results obtained at (I) and (II); wherein the following is indicative of efficacy of treatment:
(a) I_{sc cAMP} is lower in (I) than (II);
(b) I_{sc ENAC} is higher in (I) than (II);
(c) I_{sc basal} is higher in (I) than (II); and/or
(d) ΔVₜₑ is higher in (I) than (II).

The invention further provides a method of assaying for the efficacy of a test agent for treatment of cystic fibrosis, the method comprising:

(i) measuring in a test sample of human nasal epithelial cells (HNEC) from a cystic fibrosis patient:
(a) the cAMP dependent component of the basal short circuit current (I_{sc cAMP}); and optionally
(b) the epithelial Na⁺ channel (ENaC) dependent component of the basal short circuit current (I_{sc ENAC}); and optionally
(c) the basal short circuit current (I_{sc basal});
and optionally
(d) the transepithelial potential difference (ΔVₜₑ)

wherein said sample is (I) a sample from a patient prior to or during treatment with said test agent, and (II) a sample or samples taken from said patient at a later time point during said treatment, or after receiving said treatment; and comparing the results obtained at (I) and (II); wherein the following is indicative of efficacy of said test agent for treatment of cystic fibrosis:
(a) I_{sc cAMP} is lower in (I) than (II);
(b) I_{sc ENAC} is higher in (I) than (II);
(c) I_{sc basal} is higher in (I) than (II); and/or
(d) ΔVₜₑ is higher in (I) than (II).

In some embodiments, said cystic fibrosis is atypical cystic fibrosis. Atypical cystic fibrosis is described above and is characterized by, for example, normal or intermediate range sweat chloride level and only one or no identified CF-causing mutations.

The present invention also relates to a method of monitoring the efficacy of treatment of cystic fibrosis, the method comprising:
(i) measuring in a test sample of human nasal epithelial cells (HNEC) from a cystic fibrosis patient:
   (a) the cAMP dependent component of the basal short circuit current (I_{sc cAMP}),
   (b) optionally, the epithelial Na⁺ channel (ENaC) dependent component of the basal short circuit current (I_{sc ENAC}),
   (c) optionally, the basal short circuit current (I_{sc basal}), and
   (d) optionally, the transepithelial potential difference (ΔVₜₑ),
   wherein said sample is (I) a sample from said patient prior to or during said treatment, and (II) a sample or samples taken from said patient at a later time point, during said treatment or after receiving said treatment; and
(ii) comparing the results obtained at (I) and (II),
   wherein the following is indicative of efficacy of treatment for a cystic fibrosis patient with a classic phenotype:
      (a) I_{sc cAMP} is lower in (I) than (II),
      (b) I_{sc ENAC} is higher in (I) than (II),
      (c) I_{sc basal} is higher in (I) than (II), and/or
      (d) ΔVₜₑ is higher in (I) than (II),
   wherein an I_{sc cAMP} lower in (I) than (II) is indicative of efficacy of treatment for a cystic fibrosis patient with an atypical phenotype associated with abnormal chloride transport,
   and
   wherein an I_{sc ENAC} higher in (I) than (II) is indicative of efficacy of treatment for a cystic fibrosis patient with an atypical phenotype associated with abnormal sodium transport.

The present invention also relates to a method of assaying for the efficacy of a test agent for treatment of cystic fibrosis, the method comprising:
(i) measuring in a test sample of human nasal epithelial cells (HNEC) from a cystic fibrosis patient:
   (a) the cAMP dependent component of the basal short circuit current (I_{sc cAMP}),
   (b) optionally, the epithelial Na⁺ channel (ENaC) dependent component of the basal short circuit current (I_{sc ENAC}),
   (c) optionally, the basal short circuit current (I_{sc basal}), and
   (d) optionally, the transepithelial potential difference (ΔVₜₑ),
   wherein said sample is (I) a sample from a patient prior to or during treatment with said test agent, and (II) a sample or samples taken from said patient at a later time point, during said treatment or after receiving said treatment; and
(ii) comparing the results obtained at (I) and (II);
   wherein the following is indicative of efficacy of said test agent for treatment of cystic fibrosis with a classic phenotype:
      (a) I_{sc cAMP} is lower in (I) than (II),
      (b) I_{sc ENAC} is higher in (I) than (II),
      (c) I_{sc basal} is higher in (I) than (II), and/or
      (d) ΔVₜₑ is higher in (I) than (II),
   wherein an I_{sc cAMP} lower in (I) than (II) is indicative of efficacy of said test agent for treatment of cystic fibrosis with an atypical phenotype associated with abnormal chloride transport,
   and
   wherein an I_{sc ENAC} higher in (I) than (II) is indicative of efficacy of said test agent for treatment of cystic fibrosis with an atypical phenotype associated with abnormal sodium transport.

Method for Selecting an Agent Useful for the Treatment of Cystic Fibrosis

The present invention also relates to a method for selecting an agent useful for the treatment of cystic fibrosis, the method comprising:
(i) incubating a test sample of human nasal epithelial cells (HNEC) from a cystic fibrosis patient with a test agent,
(ii) measuring in the test sample obtained in step (i):
   (a) the cAMP dependent component of the basal short circuit current (I_{sc cAMP}),
   (b) optionally, the epithelial Na⁺ channel (ENaC) dependent component of the basal short circuit current (I_{sc ENAC}),
   (c) optionally, the basal short circuit current (I_{sc basal}), and
   (d) optionally, the transepithelial potential difference (ΔVₜₑ),
   and
(iii) selecting an agent having at least one of the properties selected in the group consist of:
   (a) increasing I_{sc cAMP},
   (b) decreasing I_{sc ENAC},
   (c) decreasing I_{sc basal}, and
   (d) decreasing ΔVₜₑ.

The expression “an agent increasing I_{sc cAMP}” herein means that the I_{sc cAMP} measured in the presence of said test agent is higher than the I_{sc cAMP} measured in the absence of said test agent.

The expression “an agent reducing I_{sc ENAC}/I_{sc basal}/ΔVₜₑ” means that the I_{sc ENAC}/I_{sc basal}/ΔV_{te cAMP} measured in the presence of said test agent is lower than the I_{sc ENAC}/I_{sc basal}/ΔV_{te cAMP} measured in the absence of said test agent.

The agent selected in step (iii) may have at least two or at least three properties selected in the group consist of:
(a) increasing I_{sc cAMP},
(b) reducing I_{sc ENAC},
(c) reducing I_{sc basal}, and
(d) reducing ΔVₜₑ.

The agent selected is step (iii) may be an agent that increases I_{sc cAMP}, reduces I_{sc ENAC}, reduces I_{sc basal} and reduces ΔVₜₑ.

The test sample measured in the presence of the test agent may originate from the same patient than the test sample measured in the absence of the test agent or from a different patient having similar values of I_{sc cAMP}, I_{sc ENAC}, I_{sc basal} and/or ΔVₜₑ.

The test sample measured in the presence of the test agent preferably originates from the same patient than the test sample measured in the absence of the test agent.

Method for Treating Cystic Fibrosis

The present invention also relates to a method for treating cystic fibrosis in a patient, the method comprising:
performing the method of diagnosis as defined above, and
when deducing said patient suffers from cystic fibrosis, administering a suitable treatment to said patient.

A suitable treatment of cystic fibrosis may comprise a treatment for preventing and/or treating a lung infection, a treatment for loosening and/or removing thick and/or sticky mucus from the lungs, a treatment for preventing and/or treating a blockage in the intestines, a high calorie and/or protein diet, a treatment for preventing dehydration and their combinations.

For example, a suitable treatment may comprise at least one anti-cystic fibrosis agent selected in the group consisting of a CFTR corrector or potentiator, an osmotic agent, an antioxidant drug, a modifier of mucus, a bronchodilator, an anti-infective compound, an anti-inflammatory drug, and bisphosphonate.

Non-limiting examples of CFTR corrector or potentiator are ivacaftor (such as Kalydeco), VX-770, VX-661 and/or VX-809.

The osmotic agent may be mannitol (such as Bronchitol).

The modifier of mucus may be selected among dornase alfa (such as Pulmozyme) and/or acetylcysteine (for example Mucomyst).

Non-limiting examples of bronchodilators are salbutamol (such as Ventolin) and/or salmeterol xinafoate (such as Serevent).

Non-limiting examples of anti-infective compounds are tobramycin (such as TOBI), azithromycin and/or josamycin (such as Josacine).

Non-limiting anti-inflammatory drugs are ibuprofen, glucocorticoids (such as dexamethasone), zileuton (for example Zyflo) and/or accolate.

Measurement of Ion Transport

Ion transport of epithelial cell membranes may be assayed by any method known in the art. The most commonly used method involves use of an Ussing chamber, which measures the short-circuit current as an indicator of net ion transport across an epithelium. The chamber is divided in two by a layer of epithelial cells, either in the form of sheets of mucosa or a monolayer of cells grown on a support. The apical (mucosal) side of the epithelium is thus separated from the basolateral side of the epithelium, and the two halves of the chamber represent respectively the apical and basolateral sides of the epithelium.

The two halves of the chamber are filled with equal amounts of an isotonic solution, such as Ringer's solution. Ion transport across the epithelium produces a potential difference across the epithelium (herein called Vₜₑ or ΔVₜₑ), which is measured using two voltage electrodes close to the epithelium. Said transepithelial potential difference (herein called Vₜₑ or ΔVₜₑ) is measured before short-circuiting the epithelium.

The short-circuit current (I_{sc}), which represents net ion transport across the epithelium, is measured by injecting a current using a pair of current electrodes located further away from the epithelium to short-circuit the epithelium.

The short-circuit current (I_{sc}) is preferably measured in an isotonic solution, preferably an isotonic solution comprising chloride ions, such as the Ringer solution, optionally in the presence of one or more specific compounds such as amiloride, forskolin IBMX and/or isoproterenol.

The isotonic solution preferably comprises from 100 mmol/l to 120 mmol/l of chloride ions, for example 109 mmol/l of chloride ions.

The injected current is adjusted to keep Vₜₑ at 0 mV. At intervals, the voltage is clamped to values different to 0 mV thus enabling an estimate of transepithelial resistance (Rₜₑ). The short circuit current is calculated as I_{sc}=Vₜₑ/Rₜₑ.

The different components of the short-circuit current may likewise be assayed by any method known in the art.

For example, the basal short circuit current (I_{sc basal}) is the I_{sc} measured in an isotonic solution, more preferably in an isotonic solution comprising chloride ions, such as the Ringer solution, without addition of any other compound, more particularly in the absence of amiloride, forskolin, IBMX and isoproterenol.

For example, the epithelial Na⁺ channel (ENAC) dependent component of the basal short circuit current (I_{sc ENAC}) may be assayed by measuring the amiloride sensitive component of the basal short circuit current. In a preferred embodiment, I_{sc ENAC} is thus equal to I_{sc amil}. Briefly, I_{sc} is allowed to stabilize and amiloride applied to the apical solution before again measuring I_{sc}. The amiloride sensitive component of I_{sc} is calculated as the difference between I_{sc} measured in the presence and absence of amiloride.

The cAMP dependent component of the basal short circuit current (I_{sc cAMP}) may be assayed by stimulation with at least one CFTR activator, for example one CFTR activator or a combination of two CFTR activators. The stimulation with at least one CFTR activator is preferably preceded by an inhibition of the sodium channels, for example with amiloride.

The CFTR activator is for example selected among isoproterenol or the combination of forskolin and IBMX. Preferred CFTR activators used to measure I_{sc cAMP} is the combination of forskolin and IBMX.

For example, the cAMP dependent component of the basal short circuit current (I_{sc cAMP}) may be assayed by stimulation with forskolin and IBMX (I_{sc forsk+IBMX}), said stimulation with forskolin and IBMX being preferably performed after an inhibition of sodium channels, for example with amiloride. In a preferred embodiment, I_{sc cAMP} is thus equal to I_{sc forsk+IBMX}. Briefly, amiloride-treated cells are stimulated with forskolin and IBMX at the basolateral side to induce cAMP-dependent Cl⁻ secretion (I_{sc forsk+IBMX}). I_{sc forsk+IBMX} is calculated as the difference between the initial value of I_{sc} preferably measured after amiloride addition and the peak value obtained in response to drug addition.

Nasal Brushing and Cell Culture

The test sample of cells is preferably a sample of nasal epithelial cells, in particular of human nasal epithelial cells (HNEC). Said HNEC cells may be obtained by surgery under local anaesthetic, but the inventors have also found that sufficient cells may also be obtained only by nasal brushing. Nasal brushing is a technique developed for harvesting nasal epithelial cells (HNEC) for studies of ciliary structure and function (Rutland and Cole, 1990, Lancet 13: 564-5), involving brushing of the inferior nasal turbinate to dislodge adherent epithelium. It is commonly used to collect HNEC for histological diagnosis of various diseases, including PCD (primary ciliary dyskinesia).

In a preferred embodiment, the nasal epithelial cells of the test sample are obtained by nasal brushing, preferably by nasal brushing of the middle turbinate and/or of the middle third of the lower turbinate.

Nasal brushing of the middle turbinate and/or of the middle third of the lower turbinate indeed allows collecting a majority of non altered nasal epithelial cells and in an adequate quantity, for example at least 500 000 nasal epithelial cells, preferably at least 700 000 nasal epithelial cells. At least one or at least two millions of nasal epithelial cells may be obtained by nasal brushing.

More preferably, said nasal epithelial cells of the test sample are nasal epithelial cells obtained by culturing at air-liquid interface nasal epithelial cells obtained from an individual by nasal brushing.

The inventors have succeeded in culturing HNEC cells obtained by nasal brushing in order to obtain substantial numbers of cells for analysis. In brief, the cells are cultured in immersion culture (usually for 24 hours) and then cultured at an air-liquid interface. The cells may be cultured at air-liquid interface for at least 5, 6, 7, 8, 9, 10, 11, 12, 13, 14, 15, 16, 17, 18, 19, 20 or at least 21 days prior to assay. Such long-term culture may permit larger quantities of differentiated cells to be obtained. Typically, the first week in culture is characterized by rapid proliferation of the cells, with the start of some signs of differentiation, while during the second week the cells stabilize and differentiate such that ciliary, basal and secretory cells may be observed. The epithelial identity of the cells in culture may be confirmed by detection of expression of an epithelial cell marker such as cytokeratin for basal cells, MUC5AC for mucous cells and tubulin for ciliated cells.

The expressions “mucous cells”, “secretory cells” and “calciform cells” are herein synonymous.

The present invention thus relates to a method for preparing human nasal epithelial cells from a cell sample obtained by nasal brushing, comprising:
culturing said cell sample in an immersion culture, preferably for 24 hours, and
culturing said cell sample at air-liquid interface, preferably for at least 6 days.

The cell sample may be cultured at air-liquid interface for at least 6, at least 7, at least 8, at least 9, at least 10, at least 11, at least 12, at least 13, at least 14, at least 15, at least 16, at least 17, at least 18, at least 19, at least 20 or at least 21 days.

For example, the cell sample is cultured at air-liquid interface for 6, 7, 8, 9, 10, 11, 12, 13, 14, 15, 16, 17, 18, 19, 20 or 21 days.

The steps of culture are preferably performed at 37° C. and in 5% CO₂.

The culture medium used in the immersion culture and/or in the culture at an air-liquid interface is, preferably, supplemented with at least one antibiotic, fetal calf serum and/or at least one cell activator. Said culture medium is, for example DMEM/F12, preferably supplemented with at least one antibiotic, fetal calf serum and/or at least one cell activator.

DMEM/F12 comprises 15 mM HEPES and sodium bicarbonate with pyridoxine, supplemented with 0.365 gm/L L-glutamine. DMEM/F12 is a 1:1 mixture of DMEM and Ham's F-12. This formulation combines DMEM's high concentrations of glucose, amino acids and vitamins with F-12's wide variety of components. DMEM/F12 contains no proteins, lipids or growth factors.

A combination of antibiotics that may be used in the culture medium comprises or consists in penicillin, streptomycin, amphotericin B and/or gentamicin. For example, a combination of antibiotics that may be used in the culture medium comprises or consists in 100 U/ml of penicillin, 100 mg/ml of streptomycin, 2.5 μg/ml of amphotericin B and/or 100 mg/ml of gentamicin.

Cell activators are for example a serum substitute for animal cell culture, such as Ultroser G, preferably used at a concentration of 2%.

The present invention particularly relates to a method for preparing nasal epithelial cells from a human cell sample obtained by nasal brushing, comprising the following steps:
optionally washing the cells of said cell sample, for example in a culture medium preferably comprising at least one antibiotic,
optionally suspending the cells in a trypsin-ethylenediamine tetra-acetic acid (EDTA) solution, preferably a 0.25% trypsin-ethylenediamine tetra-acetic acid (EDTA) solution, preferably for 2 minutes,
incubating the cells in a culture medium, preferably comprising at least one antibiotic, fetal calf serum and/or at least one cell activator, such as DMEM/F12 supplemented with antibiotics, 10% fetal calf serum and preferably cell activators, preferably for 5 minutes,
plating cells on a support, preferably a permeable polycarbonate support coated with type IV collagen, for example at a density of 750 000 cells/cm²,
culturing said cells in an immersion culture, preferably at 37° C. in 5% CO₂, preferably for 24 hours,
culturing said cells at an air-liquid interface, preferably for at least 6, at least 7, at least 8, at least 9, at least 10, at least 11, at least 12, at least 13, at least 14, at least 15, at least 16, at least 17, at least 18, at least 19, at least 20 or at least 21 days.

In a preferred embodiment, the method for preparing nasal epithelial cells does not comprise a step of suspending the cells in a trypsin-ethylenediamine tetra-acetic acid (EDTA) solution.

The sooner the cells are processed after nasal brushing, the better the results of said method of preparing nasal epithelial cells are.

Thus, in a preferred embodiment, the step of incubating the cells in a culture medium is performed at most 1 hour after nasal brushing, preferably at most 30 minutes after nasal brushing.

The nasal epithelial cells obtained by the above method proliferate and differentiate in a pseudo-stratified epithelium comprising ciliated cells, basal cells and mucous cells. Said nasal epithelial cells express CFTR at their apical pole. Besides, said ciliated cells express βIV tubulin, said basal cells express cytokeratin 14 and said mucous cells express mucin MUC5AC.

Said test sample may thus be, for example,

(i) a sample of HNEC cells obtained from an individual suspected of suffering from cystic fibrosis, or

(ii) a sample of HNEC cells obtained by culturing HNEC cells obtained from said individual.

Controls

The I_{sc} and ΔVₜₑ values measured in the test sample may be compared to a normal control value or range of values in order to effect a diagnosis.

A normal control value or range of values may be obtained by, for example, assaying said values in cells taken from a normal subject or group of subjects (for instance healthy subjects with no symptoms of CF) or a randomly selected group of subjects and obtaining an average or median figure. Said normal control value or range of values may then be fixed equal to, lower or higher than the values or the average of values measured in said cells.

Said normal control may be, for example, a predetermined value or range of values. A measured value of I_{sc basal}, I_{sc ENAC} and/or I_{sc cAMP} lying above or below the normal control value or the normal range may be diagnostic of cystic fibrosis. For example, a diagnosis may be made if I_{sc basal} is higher in the test sample than the normal control value or the normal control range; and I_{sc ENAC} is higher in the test sample than the normal control value or the normal control range; and/or I_{sc cAMP} is lower in the test sample than the normal control value or the normal control range and/or ΔVₜₑ is higher in the test sample than the normal control value or the normal control range.

A normal control value of I_{sc basal} may be, for example, about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 μA/cm².

In a preferred embodiment, a normal control value of I_{sc basal} may be, for example, about 20, 21, 22, 23, 24, 25, 26, 27, 28, 29, 30, 31, 32, 33, 34, 35, 36, 37, 38, 39 or 40 μA/cm².

A normal control value of I_{sc ENAC} may be, for example, about 20, 21, 22, 23, 24, 25, 26, 27, 28, 29, 30, 31, 32, 33, 34, 35, 36, 37, 38, 39, or 40 μA/cm².

In a preferred embodiment, a normal control value of I_{sc ENAC} may be, for example, about 20, 21, 22, 23, 24, 25, 26, 27, 28, 29 or 30 μA/cm²

A normal control value of I_{sc cAMP} may be, for example, about 0.5, 0.6, 0.7, 0.8, 0.9, 1.0, 1.1, 1.2, 1.3, 1.4, 1.5, 1.6, 1.7, 1.8, 1.9, 2.0, 2.5, 3.1, 3.5, 4.0, 4.5, 5, 6, 7, 8, 9, 10, 11, 12, 13, 14, 15, 16, 17, 18, 19 or 20 μA/cm².

In a preferred embodiment, a normal control value of I_{sc cAMP} may be, for example, about 2.0, 2.5, 3.1, 3.5, 4.0, 4.5, 5, 6, 7, 8, 9, 10, 11, 12, 13, 14, 15, 16, 17, 18, 19 or 20 μA/cm².

A normal control value of ΔVₜₑ may be, for example, about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mV.

In a preferred embodiment, a normal control value of ΔVₜₑ may be, for example, about 10, 11, 12, 13, 14, 15, 16, 17, 18, 19, 20, 21, 22, 23, 24, 25, 26, 27, 28, 29 or 30 mV.

The lower end of said normal range of I_{sc basal}, I_{sc ENAC} or I_{sc cAMP} may be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 μA/cm².

In a preferred embodiment, the lower end of said normal range of I_{sc basal}, I_{sc ENAC} or I_{sc cAMP} may be, for example 2, 3, 4, 5, 6, 7, 8, 9, 10, 11, 12, 13, 14, 15, 16, 17, 18, 19, 20, 21, 22, 23, 24, 25, 26, 27, 28, 29 or 30 μA/cm².

The upper end of said normal range of I_{sc basal}, I_{sc ENAC} or I_{sc cAMP} may be, for example, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, 41, 42, 43, 44, 45, 46, 47, 48, 49, 50, 51, 52, 53, 54, 55, 56, 57, 58, 59 or 60 μA/cm².

In a preferred embodiment, the upper end of said normal range of I_{sc basal}, may be, for example, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 μA/cm².

In a preferred embodiment, the upper end of said normal range of I_{sc ENAC} may be, for example, 10, 11, 12, 13, 14, 15, 16, 17, 18, 19, 20, 21, 22, 23, 24, 25, 26, 27, 28, 29 or 30 μA/cm².

The term ‘about’ as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The invention will now be described in more detail by means of the following non-limiting examples. All references cited herein, including journal articles or abstracts, published or unpublished patent applications, issued patents or any other references, are hereby incorporated by reference in their entirety, including all data, tables, figures and text presented in the cited references.

### EXAMPLES

### Example 1

### Bioelectric Characteristics of HNECs Cultured at Air-Liquid Interface in Healthy Individuals Versus in CF Patients

Materials and Methods

Primary Cultures of Human Nasal Epithelial Cells

Human nasal epithelial cells were collected from 5 healthy individuals and 5 cystic fibrosis patients. Said cystic fibrosis patients had a classic phenotype. All participants had been previously subjected to a complete scanning of the coding sequences and a search for large rearrangements following our routine analysis on gDNA to look for CFTR gene mutations. Under nasal endoscopy, after local anesthesia with a cotton pellet soaked in lidocaine (3.4%) and naphazolin (0.02%), HNEC were collected from the nasal epithelium by gently brushing the inferior turbinates. This protocol was approved by the Institutional Review Board and ethics committee of our institution (CPP, Ile de France IX), and informed consent was obtained from all participants. HNEC samples were immediately placed in DMEM/F12 supplemented with antibiotics (100 U/ml of penicillin, 100 mg/ml of streptomycin, 2.5 μg/ml of amphotericin B and 100 mg/ml of gentamicin) and transported to the laboratory. After centrifugation (1,500 rpm, 5 minutes), HNEC were suspended in 0.25% trypsin-ethylenediamine tetra-acetic acid (EDTA) solution for 2 minutes and incubated in DMEM/F12-antibiotics with 10% foetal calf serum. Finally, HNEC were plated on permeable polycarbonate supports (Snapwell®, Costar, Cambridge, USA) (750 000 cells/cm²) for short-circuit measurements. All inserts had a diameter of 12-mm and were coated with type IV collagen. HNEC were incubated at 37° C. in 5% CO₂. For the first 24 hours, HNEC were incubated with 1 ml of DMEM/F12-antibiotics with 2% Ultroser G outside the insert and DMEM/F12-antibiotics with 10% FCS inside the insert. After 24 hours, medium was removed inside the inserts in order to place the cells at an air-liquid interface, and medium outside the inserts was then changed daily. Transepithelial resistance and transepithelial potential difference were measured every three days using a microvoltmeter (World Precision Instruments, Astonbury, UK). Experiments were performed at day 14 after isolation.

Electrophysiological Studies

Measurements of short-circuit current (I_{sc}), transepithelial potential difference (ΔVte), and transepithelial resistance (Rte) were performed in Snapwell inserts mounted in vertical diffusion chambers and bathed with Ringer solution (pH 7.4) continuously bubbled with 5% CO₂-95% air at 37° C. The apical and basolateral chambers were filled with (in mM): 137 NaCl, 5.6 KCl, 1.9 CaCl₂, 1.2 MgCl₂, 5.9 CH₃COONa, 1.3 NaH₂PO₄, 10 HEPES and 10 glucose. PD was short-circuited to 0 mV with a voltage clamp (World Precision Instruments, Astonbury, UK) connected to the apical and basolateral chambers via Ag—AgCl electrodes and agar bridges in order to measure I_{sc} by Ohm's law. Isc was allowed to stabilize, before adding the drugs. Amiloride (10⁻⁴M) was applied to the apical solution to calculate the amiloride sensitive part of I_{sc} (I_{sc amil}), which is the difference between I_{sc} measured in the absence and presence of amiloride. Amiloride treated HNEC were then stimulated with forskolin (10⁻⁵ M, basolateral side) and IBMX (10⁴M, basolateral side) to induce cAMP-dependent Cl-secretion (Isc IBMX−forsk). I_{sc} IBMX−forsk was the difference between the initial value of I_{sc} and the peak value obtained in response to drug addition.

Results

This study highlights for the first time that HNEC collected by brushing the inferior turbinates can be cultured at air liquid interface at least for 14 days. A mean of 800 000±210 000 cells were collected in healthy individuals and CF patients.

The nasal epithelial cells proliferate and differentiate in a pseudo-stratified epithelium comprising ciliated cells, basal cells and mucous cells. Immunofluorescence studies have shown that the nasal epithelial cells express CFTR at their apical pole, the ciliated cells βIV tubulin, the basal cells cytokeratin 14 and the mucous cells mucin MUC5.

Second, short-circuit current measurements in Ussing chambers were possible in each samples even in CF patients. Similar results for transepithelial resistance (Rte) were obtained in CF HNEC (846±83.9 Ω·cm²) compared to healthy individuals HNEC (844.6±85.3 Ω·cm²). Transepithelial potential difference and basal Isc were significantly increased in CF HNEC (48.4±10.1 mV and 57.1±9.1 μA/cm² respectively) compared to healthy individuals HNEC (24.2±4.1 mV and 28.7±5.4 μA/cm² respectively) (p<0.05). Isc amil was largely and significantly increased in HNEC from CF patients (43.6±7.7 μA/cm²) compared to healthy individuals HNEC (14.4±4.5 μA/cm²) (p<0.05). Isc IBMX+forsk was significantly decreased in CF HNEC (0.9±0.3 μA/cm²) compared to healthy individuals HNEC (7.5±1.51 μA/cm²).

Table 1 shows the transepithelial resistance (Rte), transepithelial potential difference, and short circuit current (Isc) measured using a voltage-clamp system as described above. HNEC grown for 14 days on Snapwell filters were exposed to Amiloride (10⁻⁴M) at apical side to calculate the amiloride sensitive part of Isc (Isc amil). Amiloride treated HNEC were then stimulated with forskolin (10⁻⁵ M, basolateral side) and IBMX (10⁴M, basolateral side) to induce cAMP-dependent Cl-secretion (Isc IBMX−forsk).

TABLE 1
Bioelectric measurements in HNEC cultured at air-liquid interface in
healthy individuals and CF patients

| | | Potential | Basal | | |
|---|---|---|---|---|---|
| | Rte | difference | Isc | Isc amil | Isc IBMX-Forsk |
| | Ω · cm² | mV | μA/cm² | μA/cm² | μA/cm² |
| Healthy | 847 | 19.5 | 23 | 13 | 6 |
| individual 1 | | | | | |
| Healthy | 948 | 27.6 | 29.1 | 15.2 | 7 |
| individual 2 | | | | | |
| Healthy | 776 | 29.2 | 37.6 | 21.9 | 7.8 |
| individual 3 | | | | | |
| Healthy | 751 | 20.9 | 27.8 | 11.4 | 10 |
| individual 4 | | | | | |
| Healthy | 908 | 23.8 | 26.2 | 10.5 | 7 |
| individual 5 | | | | | |
| CF patient 1 | 784 | 53.3 | 67.9 | 55 | 1.5 |
| CF patient 2 | 851 | 50.5 | 59.3 | 42.7 | 0.6 |
| CF patient 3 | 974 | 61.4 | 63 | 46.7 | 0.9 |
| CF patient 4 | 753 | 35.4 | 47 | 39.2 | 1.1 |
| CF patient 5 | 861 | 41.8 | 48.5 | 34.8 | 0.8 |

### Example 2

### Diagnostic Method According to the Invention Versus Measurement of Nasal Transepithelial Potential Difference (NPD)

Materials and Methods

Primary Cultures of Human Nasal Epithelial Cells and Electrophysiological Studies

Primary cultures of human nasal epithelial cells were obtained as described in example 1 from cell samples obtained by nasal brushing in nine CF atypical patients, ten classic CF patients and ten healthy individuals.

Atypical CF patients had normal or intermediate sweat test (between 30 and 59 mmol/l) and/or one (ΔF508 class II mutation, IV S8/5T class V mutation) or no identified CF-causing mutation.

A mean of 950 000±200 000 cells were collected in each patient or healthy individual. Short-circuit current measurements in Ussing chambers were performed in each sample, as described in example 1.

Nasal Transepithelial Potential Difference (DPN) Measurements

An electrode was inserted into the nasal mucosa and a second electrode into the arm of an individual. Baseline PD (Potential Difference) was measured after perfusion of the nasal epithelium with Ringer saline solution. PD changes were recorded after perfusion with 100 mM amiloride in saline solution to block Na+ current (referred to as ΔAmiloride) and then after 100 mM amiloride plus 10 mM isoproterenol in a Cl⁻-free solution, to stimulate PKA-dependent CFTR-related Cl⁻ conductance (referred to as ΔLowCl⁻Iso).

Results

In order to confirm the validity of the diagnostic method starting from cells obtained by nasal brushing, the results were compared to the results obtained by DPN measurements (cf. Table 2).

Similar results were obtained for transepithelial resistance (Rte) in atypical CF HNEC (748.6+/−60.9 Ω·cm²) compared to classic CF HNEC (846±83.9 Ω·cm²) and healthy individuals HNEC (844.6±85.3 Ω·cm²). Transepithelial potential difference and basal Isc were significantly different in atypical CF HNEC (15.8+/−3.5 mV and 24.18±7.8 μA/cm² respectively) compared to classic CF HNEC (48.4±10.1 mV, 57.1±9.1 μA/cm²) (p<0.05) even though currents were similar in healthy individuals HNEC (24.2±4.1 mV and 28.7±5.4 μA/cm² respectively). Isc amil was significantly different in atypical CF HNEC (12.85±1.98 μA/cm²) compared to HNEC from classic CF patients (43.6±7.7 μA/cm²) (p<0.05) but was similar to healthy individuals HNEC (14.4±4.5 μA/cm²). Isc IBMX+forsk was significantly decreased in atypical CF HNEC (1.35+/−0.59 μA/cm²) compared to healthy individuals HNEC (8.7±1.07 μA/cm²) (p<0.0001) but very close to classic CF HNEC (0.9±0.3 μA/cm²) (p=0.34).

As regards to DPN measurements, median results revealed in atypical CF patient a maximal basal PD at −22±8.7 mV, ΔAmiloride 7±3 and ΔLowCl⁻Iso 1.7±0.5. In comparison, Wilson et al. (The Journal of Pediatrics, 1997, Vol 132, Number 4) have described, in people without CF, a maximal basal PD around −24 mV, ΔAmiloride around 12 mV and ΔLowCl⁻Iso around −21 mV; in classic CF patients, a maximal basal PD and ΔAmiloride significantly higher (around −52 mV and around 35 mV, respectively) and ΔLowCl⁻Iso very low (around 2 mV).

TABLE 2
Bioelectric measurements in HNEC from nasal brushing compared to
DPN measurements

| | | |
|---|---|---|
| Atypical | Classic | Healthy |
| CF patient | CF patient | individual |

Nasal brushing results

| | | | |
|---|---|---|---|
| Transepithelial | 748.6 +/− 60.9 | 846 +/− 83.9 | 844.6 +/− 85.3 |
| resistance Rte | | | |
| (Ω · cm²) | | | |
| Transepithelial | 15.8 +/− 3.5^{a} | 48.4 +/− 10.1^{a} | 24.2 +/− 4.1 |
| potential | | | |
| difference (mV) | | | |
| Basal Isc | 24.18 +/− 7.8^{b} | 57.1 +/− 9.1^{b} | 28.7 +/− 5.4 |
| (μA/cm²) | | | |
| Isc amil | 12.85 +/− 1.98^{c} | 43.6 +/− 7.7^{c} | 14.4 +/− 4.5 |
| Isc IBMX + forsk | 1.35 +/− 0.59^{d} | 0.9 +/− 0.3 | 8.7 +/− 1.07^{d} |

DPN results

| | | | |
|---|---|---|---|
| Basal PD (mV) | −22 +/− 8.7 | −52 +/− 9* | −24 +/− 8* |
| ΔAmiloride (mV) | 7 +/− 3 | 35 +/− 10* | 12 +/− 5* |
| ΔLowCl⁻Iso | 1.7 +/− 0.5 | 2 +/− 4* | −21 +/− 9* |
| (mV) | | | |

*from Wilson et al., 1998;
^{a}p < 0.05,
^{b}p < 0.05;
^{c}p < 0.05;
^{d}p < 0.0001

These results highlight that the diagnostic method starting from cells obtained by nasal brushing is a very reliable test, since atypical CF patients have the same response in DPN test.

The I_{sc IBMX+forsk} value is sufficient to determine that an individual suffers from cystic fibrosis. High values of transepithelial potential difference, I_{sc basal} or I_{sc amil} indicate a cystic fibrosis patient with a classic phenotype. A low I_{sc IBMX+forsk} value associated with normal values of transepithelial potential difference, I_{sc basal} and I_{sc amil} indicate a cystic fibrosis patient with an atypical phenotype.

### CONCLUSIONS

The results show that the diagnostic method according to the invention is a very reliable test and allows obtaining the same response as the one obtained in DPN test. Besides, the diagnostic method is a non invasive method that can be used in any patient, whatever the physical condition of the patient is. For example, in case of inflammation, which often happens in cystic fibrosis patients, the basal DPN of said patient may be a positive value, which renders the DPN test impossible to be performed.

The diagnostic method also allows discriminating healthy individuals from cystic fibrosis patients, but also among cystic fibrosis patients those with a classic phenotype from those with an atypical phenotype.

1. A method of diagnosis of cystic fibrosis, the method comprising measuring in a test sample of human nasal epithelial cells (HNEC) the cAMP dependent component of the basal short circuit current (I_{sc cAMP}).

2. The method of diagnosis according to claim 1, comprising deducing that said test sample originates from a cystic fibrosis patient when I_{sc cAMP} is below 2 μA/cm².

3. The method of diagnosis according to claim 1, the method comprising further measuring in a test sample of human nasal epithelial cells (HNEC):
(a) the epithelial Na⁺ channel (ENAC) dependent component of the basal short circuit current (I_{sc ENAC}),
(c) the basal short circuit current (I_{sc basal}), and/or
(d) the transepithelial potential difference (ΔVₜₑ).

4. The method of diagnosis according to claim 3, the method further comprising making a diagnosis based on the measured values,
wherein a diagnosis of cystic fibrosis with a classic phenotype is made when:
(a) I_{sc cAMP} is below 2 μA/cm², and
(b) I_{sc ENAC} is above 30 μA/cm², I_{sc basal} is above 40 μA/cm² and/or ΔVₜₑ is above 30 mV, and
wherein a diagnosis of cystic fibrosis with an atypical phenotype is made:
when:
(a) I_{sc cAMP} is below 2 μA/cm², and
(b) I_{sc ENAC} is below 20 μA/cm², I_{sc basal} is below 30 μA/cm² and/or ΔVₜₑ is below 25 mV,
or when:
(a) I_{sc cAMP} is above 2 μA/cm², and
(b) I_{sc ENAC} is above 30 μA/cm², I_{sc basal} is above 40 μA/cm² and/or ΔVₜₑ is above 30 mV.

5. The method of diagnosis of cystic fibrosis according to claim 1, the method further comprising:
(i) comparing said I_{sc cAMP} measured value to a normal control, and
(ii) making a diagnosis based on said comparison, wherein a diagnosis of cystic fibrosis is made when I_{sc cAMP} is lower in the test sample than the normal control.

6. The method of diagnosis of cystic fibrosis according to claim 3, the method comprising:
(i) comparing said I_{sc} measured values, and optionally said ΔVₜₑ measured value, to a normal control, and
(ii) making a diagnosis based on said comparison,
wherein a diagnosis of cystic fibrosis with a classic phenotype is made when:
(a) I_{sc cAMP} is lower in the test sample than the normal control, and
(b) I_{sc ENAC}, I_{sc basal} and/or ΔVₜₑ is higher in the test sample than the normal control,
and
wherein a diagnosis of cystic fibrosis with an atypical phenotype is made
when:
(a) I_{sc cAMP} is lower in the test sample than the normal control, and
(b) I_{sc ENAC}, I_{sc basal} and/or ΔVₜₑ is equal or lower in the test sample than the normal control,
or when:
(a) I_{sc cAMP} is equal or higher in the test sample than the normal control, and
(b) I_{sc ENAC}, I_{sc basal} and/or ΔVₜₑ is higher in the test sample than the normal control.

7. The method according to claim 5, wherein said normal control is a predetermined value or range of values.

8. The method according to claim 1, wherein
(i) I_{sc cAMP} is assayed, after inhibition of the sodium channels, with a stimulation with forskolin and IBMX (I_{sc forsk+IBMX}); and/or
(ii) I_{sc ENAC}, where assayed, is assayed by measuring the amiloride sensitive component of the basal short circuit current (Iₐₘᵢₗ).

9. The method according to claim 1, wherein said test sample of human nasal epithelial cells (HNEC) is a sample of human nasal epithelial cells obtained from an individual suspected of suffering from cystic fibrosis.

10. The method according to claim 9, wherein said test sample of human nasal epithelial cells (HNEC) is a sample of human nasal epithelial cells obtained by culturing human nasal epithelial cells from an individual suspected of suffering from cystic fibrosis.

11. The method according to claim 10, wherein the human nasal epithelial cells (HNEC) of said test sample are human nasal epithelial cells obtained by the method according to claim 16.

12. The method according to claim 9, wherein said human nasal epithelial cells are obtained by brushing of the nasal inferior turbinates of said individual.

13. A method of monitoring the efficacy of treatment of cystic fibrosis, the method comprising:
(i) measuring in a test sample of human nasal epithelial cells (HNEC) from a cystic fibrosis patient:
(a) the cAMP dependent component of the basal short circuit current (I_{sc CAMP}),
(b) optionally, the epithelial Na⁺ channel (ENaC) dependent component of the basal short circuit current (I_{sc ENAC}),
(c) optionally, the basal short circuit current (I_{sc basal}), and
(d) optionally, the transepithelial potential difference (ΔVₜₑ), wherein said sample is (I) a sample from said patient prior to or during said treatment, and (II) a sample or samples taken from said patient at a later time point, during said treatment or after receiving said treatment; and
(ii) comparing the results obtained at (I) and (II),
wherein the following is indicative of efficacy of treatment for a cystic fibrosis patient:
(a) I_{sc cAMP} is lower in (I) than (II),
(b) I_{sc ENAC} is higher in (I) than (II),
(c) I_{sc basal} is higher in (I) than (II), and/or
(d) ΔVₜₑ is higher in (I) than (II).

14. A method of assaying for the efficacy of a test agent for treatment of cystic fibrosis, the method comprising:
(i) measuring in a test sample of human nasal epithelial cells (HNEC) from a cystic fibrosis patient:
(a) the cAMP dependent component of the basal short circuit current (I_{sc cAMP}),
(b) optionally, the epithelial Na⁺ channel (ENAC) dependent component of the basal short circuit current (I_{sc ENAC}),
(c) optionally, the basal short circuit current (I_{sc basal}), and
(d) optionally, the transepithelial potential difference (ΔVₜₑ),
wherein said sample is (I) a sample from a patient prior to or during treatment with said test agent, and (II) a sample or samples taken from said patient at a later time point, during said treatment or after receiving said treatment; and
(ii) comparing the results obtained at (I) and (II);
wherein the following is indicative of efficacy of said test agent for treatment of cystic fibrosis:
(a) I_{sc cAMP} is lower in (I) than (II),
(b) I_{sc ENAC} is higher in (I) than (II),
(c) I_{sc basal} is higher in (I) than (II), and/or
(d) ΔVₜₑ is higher in (I) than (II).

15. A method for treating cystic fibrosis in a patient, the method comprising:
performing the method of diagnosis according to claim 1, and
when deducing said patient suffers from cystic fibrosis, administering a suitable treatment to said patient.

16. A method for preparing human nasal epithelial cells from a cell sample obtained by nasal brushing, comprising:
culturing said cell sample in an immersion culture, and
culturing said cell sample at air-liquid interface.

17. A method for selecting an agent useful for the treatment of cystic fibrosis, the method comprising:
(i) incubating a test sample of human nasal epithelial cells (HNEC) from a cystic fibrosis patient with a test agent,
(ii) measuring in the test sample obtained in step (i):
(a) the cAMP dependent component of the basal short circuit current (I_{sc cAMP}),
(b) optionally, the epithelial Na⁺ channel (ENaC) dependent component of the basal short circuit current (I_{sc ENAC}),
(c) optionally, the basal short circuit current (I_{sc basal}), and
(d) optionally, the transepithelial potential difference (ΔVₜₑ),
and
(iii) selecting an agent having at least one of the properties selected in the group consist of:
(a) increasing I_{sc cAMP},
(b) decreasing I_{sc ENAC},
(c) decreasing I_{sc basal}, and
(d) decreasing ΔVₜₑ.

18. The method according to claim 7, wherein said range of values is:
(a) I_{sc cAMP} of 6-10 μA/cm²,
(b) I_{sc ENAC} of 10-22 μA/cm²,
(c) I_{sc basal} of 23-37 μA/cm², and/or
(d) ΔVₜₑ of 15-30 mV.

19. The method according to claim 6, wherein said normal control is a predetermined value or range of values.

20. The method according to claim 19, wherein said range of values is:
(a) I_{sc cAMP} of 6-10 μA/cm²,
(b) I_{sc ENAC} of 10-22 μA/cm²,
(c)_{sc basal} of 23-37 μA/cm², and/or
(d) ΔVₜₑ of 15-30 mV.

21. A method according to claim 16, wherein:
said cell sample is cultured in an immersion culture for 24 hours and/or
said cell sample is cultured at air-liquid interface for at least 6 days.
